(19) United States
(12) Patent Application Publication
(10) Pub. No.: US 2013/0184801 A1
(43) Pub. Date: Jul. 18, 2013

(54) LEAD SHAPED FOR STIMULATION AT THE BASE LEFT VENTRICLE

(71) Applicants: Mark Carlson, Calabasas, CA (US); Gene A. Bornzin, Simi Valley, CA (US)

(72) Inventors: Mark Carlson, Calabasas, CA (US); Gene A. Bornzin, Simi Valley, CA (US)

(73) Assignee: PACESETTER, INC., Sylmar, CA (US)

(21) Appl. No.: 13/350,663

(22) Filed: Jan. 13, 2012

(51) Int. Cl. A61N 1/05

(52) U.S. Cl. 607/119

(57) ABSTRACT

Disclosed herein are a variety of implantable medical leads for coupling to an implantable pulse generator and targeted stimulation of the lateral and posterior basal left ventricular region of a patient heart. As one example, the lead may include a tubular body including proximal section, an intermediate section and a distal section. The intermediate section biases into a generally S-shaped or sinusoidal-shaped configuration when the intermediate section is in a free or non-restricted state. The proximal section proximally extends from the intermediate section to a proximal end configured to electrically couple to the implantable pulse generator. The distal section biases into a generally straight linear shaped configuration when the distal section is in a free or non-restricted state.

### FIELD OF THE INVENTION

Aspects of the present invention relate to medical apparatus and methods. More specifically, the present invention relates to implantable medical leads and methods of manufacturing and implanting such leads.

### BACKGROUND OF THE INVENTION

A recent study has analyzed the impact of left ventricular (“LV”) lead stimulation sites on patient outcomes. The study classified lead position around the perimeter of the LV short axis as anterior, posterior or lateral. With respect to the LV long axis view, the lead positioning was described as apical, mid, or basal.

The study indicated apical lead positioning was most hazardous followed by mid-ventricular lead positioning and basal lead position, which had the lowest risk. The study also suggested that posterior lead positioning and lateral lead positioning are better than anterior lead positioning. Accordingly, for the best outcomes, leads should be implanted in the lateral and posterior basal regions.

The study suggested placing the lead apically does little good and may in fact be harmful because the apical location is in close proximity to the right ventricular (“RV”) lead. This close proximity promotes a sequence of activation similar to RV pacing, which is known to be detrimental.

The lateral and posterial basal locations are regions of last activation. Accordingly, pacing the lateral and posterial basal locations corrects delayed activation and promotes improves resynchronization.

There is a need in the art for implantable medical leads and methods of implantation that facilitate pacing the lateral and posterial basal locations. There is also a need in the art for methods of manufacturing such implantable medical leads.

### BRIEF SUMMARY OF THE INVENTION

A first embodiment of the present disclosure may take the form of an implantable medical lead for coupling to an implantable pulse generator and targeted stimulation of the lateral and posterior basal left ventricular region of a patient heart. The lead may include a tubular body including proximal section, an intermediate section and a distal section. The intermediate section biases into a generally S-shaped or sinusoidal-shaped configuration when the intermediate section is in a free or non-restricted state. The S-shaped or sinusoidal-shaped configuration includes multiple hump peaks having at least a most distal hump peak and a most proximal hump peak. The proximal section proximally extends from the most proximal hump peak to a proximal end configured to electrically couple to the implantable pulse generator. The distal section biases into a generally straight linear shaped configuration when the distal section is in a free or non-restricted state. The distal section distally extends from the most distal hump peak and includes multiple electrodes and a distal free end that forms an extreme distal end of the lead.

A second embodiment of the present disclosure may take the form of an implantable medical lead for coupling to an implantable pulse generator and targeted stimulation of the lateral and posterior basal left ventricular region of a patient heart. The lead may include a tubular body having a proximal section, an intermediate section and a distal section. The intermediate section biases into a generally helically coiled configuration when the intermediate section is in a free or non-restricted state. The helically coiled configuration includes multiple helical coil loops having at least a most distal loop and a most proximal loop. The proximal section proximally extends from the most proximal loop to a proximal end configured to electrically couple to the implantable pulse generator. The distal section biases into a generally straight linear shaped configuration when the distal section is in a free or non-restricted state. The distal section distally extends from the most distal loop and includes multiple electrodes and a distal free end that forms an extreme distal end of the lead.

A third embodiment of the present disclosure may take the form of an implantable medical lead for coupling to an implantable pulse generator and targeted stimulation of the lateral and posterior basal left ventricular region of a patient heart. The lead may include a tubular body having a proximal section and a distal section. The distal section biases into a generally S-shaped or sinusoidal-shaped configuration when the distal section is in a free or non-restricted state. The S-shaped or sinusoidal-shaped configuration includes multiple hump peaks having at least a most distal hump peak and a most proximal hump peak. The most distal hump peak extends into a distal termination of the S-shaped or sinusoidal-shaped configuration in the form of a distal free end that forms an extreme distal end of the lead. The distal section includes first, second, third and fourth electrodes. The first electrode is supported on the tubular body at the distal free end. The second, third and fourth electrodes are supported on the tubular body at respective hump peaks located on the same side of the distal section as the distal free end. The proximal section proximally extends from the most proximal hump peak to a proximal end configured to electrically couple to the implantable pulse generator.

A fourth embodiment of the present disclosure may take the form of an implantable medical lead for coupling to an implantable pulse generator and targeted stimulation of the lateral and posterior basal left ventricular region of a patient heart. The lead may include a tubular body having a proximal section and a distal section. The distal section biases into a generally helically coiled configuration when the distal section is in a free or non-restricted state. The helically coiled configuration includes multiple helical coil loops having at least a most distal loop and a most proximal loop. The most distal loop extends into a distal termination of the helically coiled configuration in the form of a distal free end that forms an extreme distal end of the lead. The distal section includes first, second, third and fourth electrodes. The first electrode is supported on the tubular body at the distal free end. The second, third and fourth electrodes are each supported on the tubular body at an extreme outward circumferential point of a respective loop. Each of the second, third and fourth electrodes is located on the same side of the distal section as the distal free end. The proximal section proximally extends from the most proximal loop to a proximal end configured to electrically couple to the implantable pulse generator.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1 is a diagrammatic depiction of an electrotherapy system electrically coupled to a patient heart as shown in an anterior view, a distal portion of a LV lead being implanted in the CS.

FIG. 2 is a side view of the distal portion of a first embodiment of a lead tubular body in a free or non-restricted state, the distal portion having an S-shaped or sinusoidal-shaped intermediate section and a generally straight distal section.

FIG. 3 is the same view of the distal portion of the lead tubular body depicted in FIG. 2, except the distal portion is deployed in, and confined or restricted by, a vascular body such as the CS.

FIG. 4 is a left lateral posterior view of the patient heart, the CS extending along the outer surface of the heart between the LV and LA patient left and generally anterior from the OS, the lead embodiment of FIG. 2 having its distal portion implanted in the CS and LMV.

FIG. 5 is the same view and lead embodiment as FIG. 4, except the lead distal portion is implanted in the CS and PCV.

FIG. 6 is the same view and lead embodiment as FIG. 4, except the lead distal portion is implanted in the CS and GCV.

FIG. 7 is a side view of the distal portion of a second embodiment of a lead tubular body in a free or non-restricted state, the distal portion having a helically coiled intermediate section and a generally straight distal section.

FIG. 8 is a left lateral posterior view of the patient heart, the CS extending along the outer surface of the heart between the LV and LA patient left and generally anterior from the OS, the lead embodiment of FIG. 7 having its distal portion implanted in the CS and PCV.

FIG. 9 is a side view of the distal portion of a third embodiment of a lead tubular body in a free or non-restricted state, the distal portion having an S-shaped or sinusoidal-shaped distal section that distally terminates in a free distal end forming an extreme distal end of the lead.

FIG. 10 is a left lateral posterior view of the patient heart, the CS extending along the outer surface of the heart between the LV and LA patient left and generally anterior from the OS, the lead embodiment of FIG. 9 having its distal portion implanted in the CS and GCV.

FIG. 11 is a side view of the distal portion of a fourth embodiment of a lead tubular body in a free or non-restricted state, the distal portion having helically coiled distal section that distally terminates in a free distal end forming an extreme distal end of the lead.

FIG. 12 is a left lateral posterior view of the patient heart, the CS extending along the outer surface of the heart between the LV and LA patient left and generally anterior from the OS, the lead embodiment of FIG. 11 having its distal portion implanted in the CS.

### DETAILED DESCRIPTION

Implementations of the present disclosure involve implantable medical leads 5 and methods of implantation that facilitate the targeted stimulation of the lateral and posterior basal region of the heart.

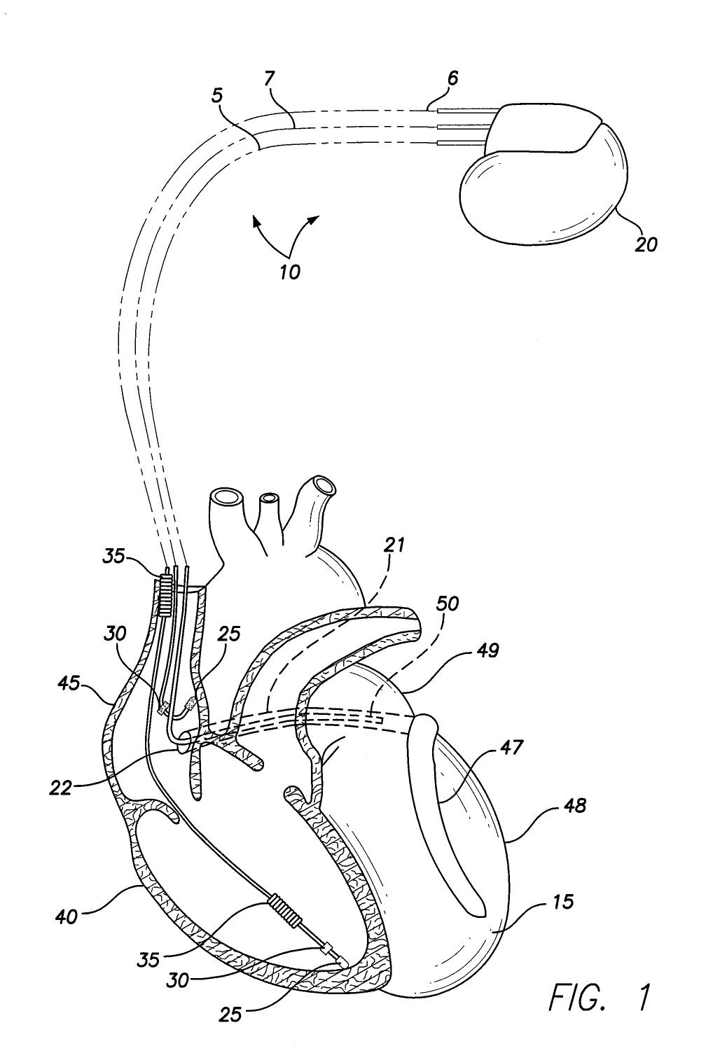

To begin a general, non-limiting discussion regarding some of the features and deployment characteristics common among the various lead and implantation embodiments disclosed herein, reference is made to FIG. 1, which is a diagrammatic depiction of an electrotherapy system 10 electrically coupled to a patient heart 15 as shown in an anterior view. As shown in FIG. 1, the system 10 includes an implantable pulse generator (e.g., pacemaker, implantable cardioverter defibrillator (“ICD”), or etc.) 20 and one or more (e.g., three) implantable medical lead 5, 6, 7 electrically coupling the patient heart 15 to the pulse generator 10. While the following discussion will focus on the configuration and implantation of the left ventricular (“LV”) lead 5 extending into the coronary sinus (“CS”) 21 via the coronary sinus ostium (“OS”) 22, it should be remembered that the system 10 may employ only the LV lead 5 or the LV lead 5 in conjunction with other leads, such as, for example, a right ventricular (“RV”) lead 6 and/or right atrial (“RA”) lead 7. The RV and RA leads 6, 7 may employ pacing electrodes 25, sensing electrodes 30 and shock coils 35 as known in the art to respectively provide electrical stimulation to the right ventricle 40 and right atrium 45 of the heart 15.

As can be understood from FIG. 1, which shows an anterior view of the patient heart 15, the CS 21 extends generally patient right to patient left from the OS 22 and, further, posterior to anterior until transitioning into the great cardiac vein 47, which then extends in a generally inferior direction along the anterior region of the left ventricle (“LV”) 48. In extending generally posterior to anterior from the OS 22 until transitioning into the great cardiac vein 47, the CS 22 is inferior to the left atrium (“LA”) 49 and superior to the LV 48.

As indicated in FIG. 1, in most embodiments of the LV lead 5 disclosed herein, the distal portion 50 of the LV lead 5 does not extend into the great cardiac vein 47, but is instead implanted in the CS 21 or vein branches extending off of the CS 21, as described in detail below. As explained in the discussion below, the distal portion 50 of each embodiment of the LV lead 5 disclosed herein is configured to facilitate the distal portion 50 being implanted in the CS 21 and, more particularly, in the lateral and posterior basal region of the heart.

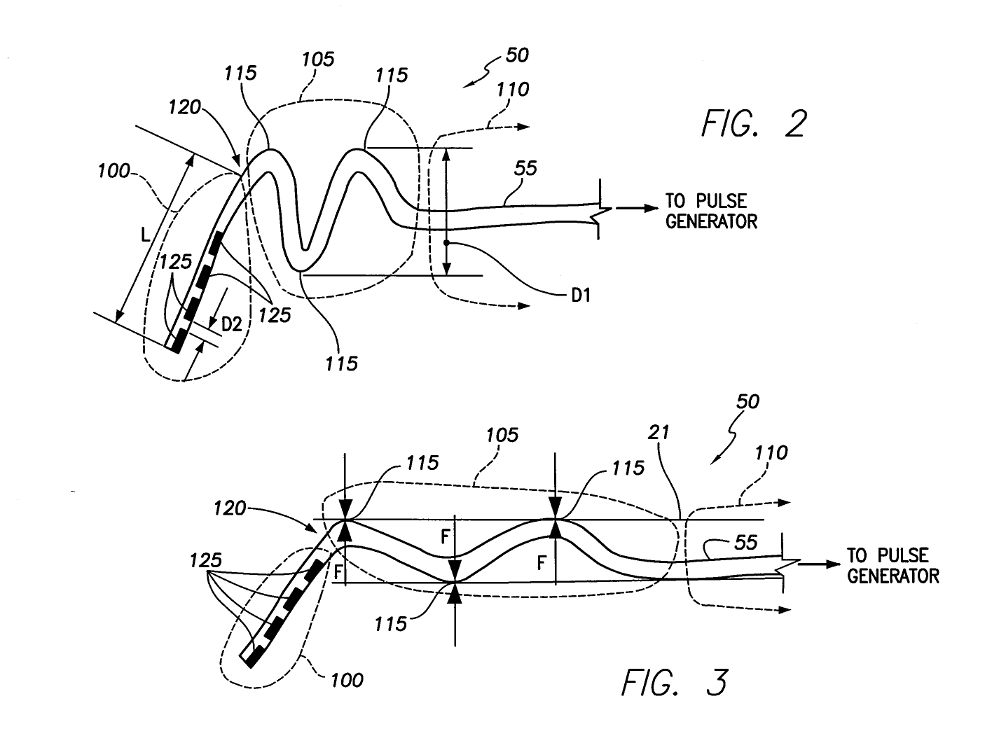

For a discussion of the configuration of the distal portion 50 of a tubular body 55 of a first embodiment of the LV lead 5, reference is made to FIGS. 2 and 3, which are side views of the distal portion 50 of the lead tubular body 55 in a free or non-restricted state and a restricted state, respectively. For purposes of discussion, when the distal portion 50 of the tubular body 55 of the LV lead 5 is said to be in a free or non-restricted state, the distal portion 50 exists in a configuration that the distal portion 50 naturally biases to absent the distal portion 50 being acted upon by an outside force such as, for example, a stylet being extended through the distal portion 50, a sheath or catheter being extended over the distal portion 50, or the distal portion 50 being confined within a vascular structure such as, for example, the CS 21.

As indicated in FIG. 2, when the distal portion 50 of the tubular body 55 of the first embodiment of the LV lead 5 is in a free or non-restricted state, the distal portion 50 includes an extreme distal section 100, an intermediate section 105 immediately proximal the extreme distal section 100, and a proximal section 110 that proximally extends from the intermediate section 105 towards the proximal end of the LV lead 5 that connects to the pulse generator 20, as can be understood from FIG. 1. The tubular body 55 of the intermediate section 105 undulates in a sinusoidal or S-shaped configuration such that the intermediate section 105 has multiple hump peaks 115 (e.g., two, three or more hump peaks 115) when in a free or non-restricted state. In a free or non-restricted state, the peak-to-peak distance D1 is between approximately 1.3 cm and approximately 1.5 cm.

In other words, as can be understood from FIG. 2, when the intermediate section 105 is in the free or non-restricted state and the proximal section 110 is laid out in a straight line, the peak-to-peak distance D1 extends generally perpendicular to a longitudinal axis of the straight proximal section 110. Further, the peak-to-peak distance D1 extends between the peak 115 of a first hump or bend and a peak 115 of a second hump or bend immediately adjacent the first hump or bend and projecting in a direction opposite the first hump or bend. Thus, when the intermediate section 105 is in the free or non-restricted state as depicted in FIG. 2, the intermediate section 105 includes a peak-to-peak distance D1 of between approximately 1.3 cm and approximately 1.5 cm.

The tubular body 55 of the intermediate section 105 has a size of between approximately seven French and approximately eight French and is formed of silicone rubber, polyurethane, or silicone rubber polyurethane copolymer (“SPC”). Additionally, the shape may be mechanically reinforced by using polymers of appropriate durometer or by using heat treated coiled conductors to reinforce the desired shape. Frequently, coiled co-radial or coaxial conductors made of MP35 stainless steel are used in cardiac leads and may be heat treated to take on a given shape. Thermoplastics like SPC or polyurethane may be shaped by applying heat to raise the temperature near or above the glass transition temperature of the material. Thermoset polymers like silicone rubber may be shaped in the uncured or partially cured state and the fully cured at an elevated temperature to mold in the desired shape. Frequently, coiled co-radial or coaxial conductors coils made of MP35 stainless steel are used in cardiac leads. These may be slide over a rigid mandrel and heat treated to take on a given shape. When preshaped conductors are placed in preshaped tubing having a common preshape, the configuration of the lead body is mechanically reinforced. Adjusting the degree of preshaping, the wall thickness of the tubing, the diameter of the wire making up the helical conductor, or the number of co-filers, co-radial conductors or coaxial conductors influences the modulus of the lead body. Thus, the desired biasing forces that the lead body establishes against the cardiac vessels may be adjusted within desired limits as well as the desired shape of the lead body.

As illustrated in FIG. 2, the tubular body 55 of the distal section 100 has a generally straight or linear shape when in a free or non-restricted state. The distal section 100 begins just distal of the most distal hump peak 115 of the intermediate section 105 and extends over a length L of between approximately 1 cm and approximately 3 cm. The tubular body 55 of the distal section 100 has a size of between approximately four French and approximately seven French and is formed of silicone rubber, polyurethane, or silicone rubber polyurethane copolymer (“SPC”). Thus, the transition 120 between the distal section 100 and intermediate section 105 may be in the form of a taper. In other words, the distal section 100 has a tubular body diameter that is smaller than a tubular body diameter of the intermediate section 105, and the lead 5 includes a tubular body diameter transition 120 between the distal section 100 and the intermediate section 105.

As can be understood from FIG. 2, when the distal section 100 and intermediate section 105 are both in the free or non-restricted state, the tubular body 55 forming the intermediate section 105 and distal section 100 generally resides in a single plane.

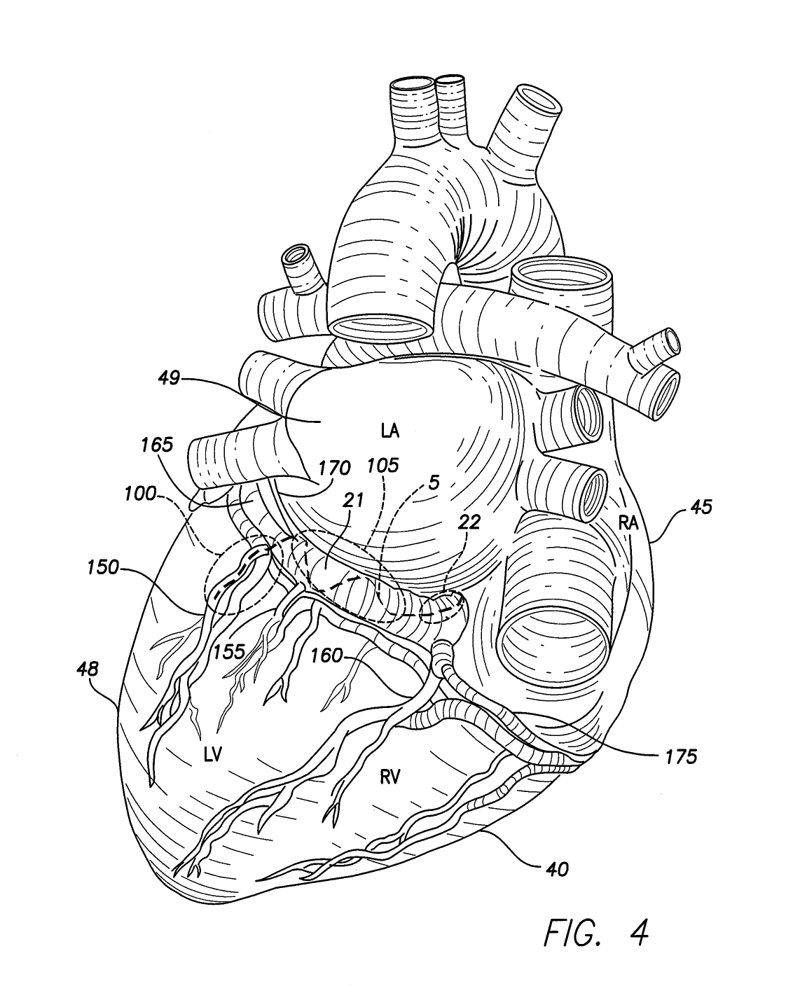

As shown in FIG. 2, the tubular body 55 of the distal section 100 supports multiple electrodes 125 (e.g., two, three, four or more electrodes 125). In one embodiment, there are four electrodes 125 forming a quadpole arrangement wherein the electrodes 125 are spaced apart from each other by a distance D2 of approximately 5 mm. In one embodiment, the electrodes may be positioned on the tubular body 55 so as to be canted (e.g., directed) towards cardiac tissue when the distal portion 50 of the LV lead 5 is deployed as discussed with respect to FIG. 4 below. For example, as can be understood from FIG. 2, in one embodiment, the multiple electrodes 125 of the distal section 100 are located on a side of the tubular body 55 generally oriented proximal. In some embodiments, the electrodes 125 are located on the distal section 100 of the tubular body such that, when looking along the length of the lead from proximal to distal, the multiple electrodes 125 are oriented between facing proximal and facing right. The electrodes 125 being so oriented on the lead body 55 results in the electrodes 125 being directed toward the cardiac tissue, which helps to avoid phrenic nerve stimulation. Also, the electrodes 125 being directed toward the cardiac tissue more efficiently directs the current to the cardiac tissue, thereby minimizing the magnitude of stimulation current needed to pace the cardiac tissue. FIG. 3 is a side view of the distal portion 50 of the LV lead 5 deployed in, and confined or restricted by, the inner wall surface of a vascular body such as the CS 21. As can be understood from FIG. 3, the body 55 of the distal portion 50 of the LV lead 5 is configured to have sufficient bias towards its free or non-restricted state depicted in FIG. 2 such that when confined in and restricted by a CS 21, each hump peak 115 exerts a force F of between approximately 13,000 dynes to approximately 36,000 dynes for each centimeter the hump peaks 115 are compressed from the free or non-restricted state peak-to-peak distance D1 of between approximately 1.3 cm and approximately 1.5 cm indicated in FIG. 2.

As shown in FIG. 4, which is a left lateral posterior view of the patient heart 15, the CS 21 extends along the outer surface of the heart 15 between the LV 48 and LA 49 patient left and generally anterior from the OS 22. A left marginal cardiac vein (“LMV”) 150, a posterior cardiac vein (“PCV”) 155 and a middle cardiac vein (“MCV”) 160 extend generally inferior off of the CS 21. A great cardiac vein (“GCV”) 165 can be seen to extend generally anterior from the CS 21, and a vein of Marshall (“VM”) 170 can be seen to extend generally anterior from the CS 21 and superior the GCV 165. A small cardiac vein (“SCV”) 175 can be seen to extend patient right and generally posterior off of the CS 21. Depending on the individual, the mid-coronary sinus, which extends from the MCV 160 to the PCV 155, may have a diameter between approximately eight millimeters and approximately ten millimeters. Depending on the individual, the distal-coronary sinus, which extends from the PCV 155 to the GCV 165, may have a diameter between approximately five millimeters and approximately seven millimeters.

As can be understood from FIG. 4, in one embodiment, the LV lead 5 can be delivered into the CS 21 and LMV 150 via one or more delivery tools (e.g., stylets, guidewires, sheaths, catheters, etc.). When the delivery tools are removed from the distal portion 50 (see FIGS. 2-3), the humped peaks 115 of the intermediate section 105 of the distal portion 50 of the LV lead 5 are free to bias against the inner wall surface of the CS 21. As a result, each hump peak 115 exerts a force F of between approximately 13,000 dynes to approximately 36,000 dynes for each centimeter the hump peaks 115 are compressed by the inner wall surface of the CS 21 from the free or non-restricted state peak-to-peak distance D1 of between approximately 1.3 cm and approximately 1.5 cm indicated in FIG. 2. The distal section 100 extends into the LMV 150 in a generally linear arrangement with the electrodes 125 oriented so as to generally face the cardiac tissue.

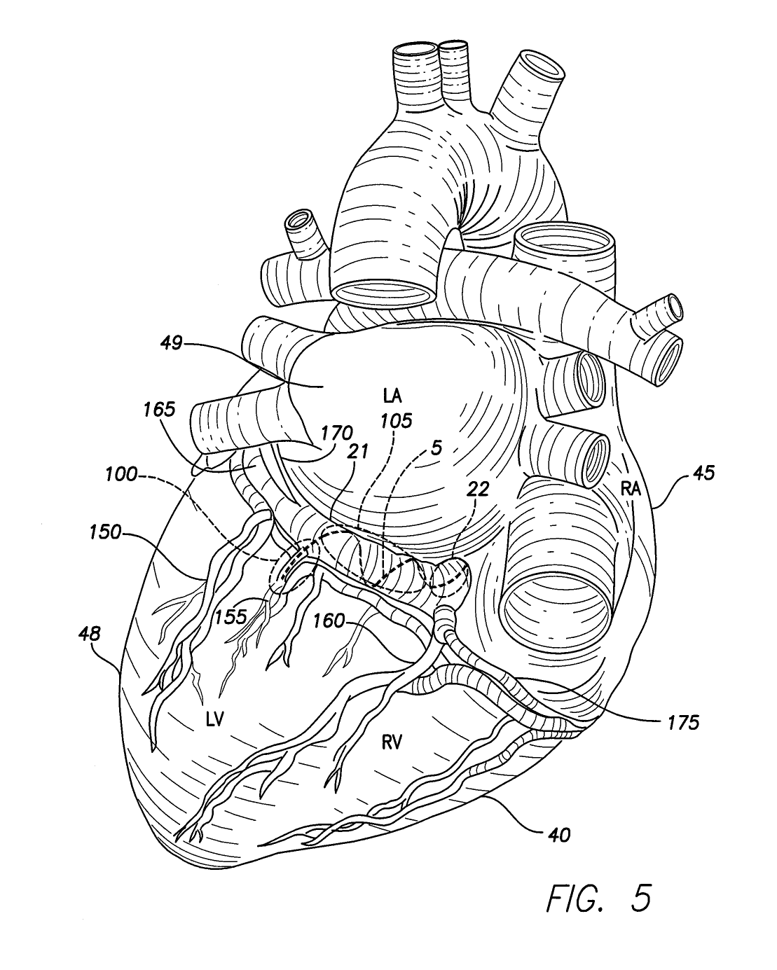

As can be understood from FIG. 5, which is the same view as FIG. 4, in one embodiment, the LV lead 5 can be delivered into the CS 21 and PCV 155 via one or more delivery tools (e.g., stylets, guidewires, sheaths, catheters, etc.). When the delivery tools are removed from the distal portion 50 (see FIGS. 2-3), the humped peaks 115 of the intermediate section 105 of the distal portion 50 of the LV lead 5 are free to bias against the inner wall surface of the CS 21. As a result, each hump peak 115 exerts a force F of between approximately 13,000 dynes to approximately 36,000 dynes for each centimeter the hump peaks 115 are compressed by the inner wall surface of the CS 21 from the free or non-restricted state peak-to-peak distance D1 of between approximately 1.3 cm and approximately 1.5 cm indicated in FIG. 2. The distal section 100 extends into the PCV 155 in a generally linear arrangement with the electrodes 125 oriented so as to generally face the cardiac tissue.

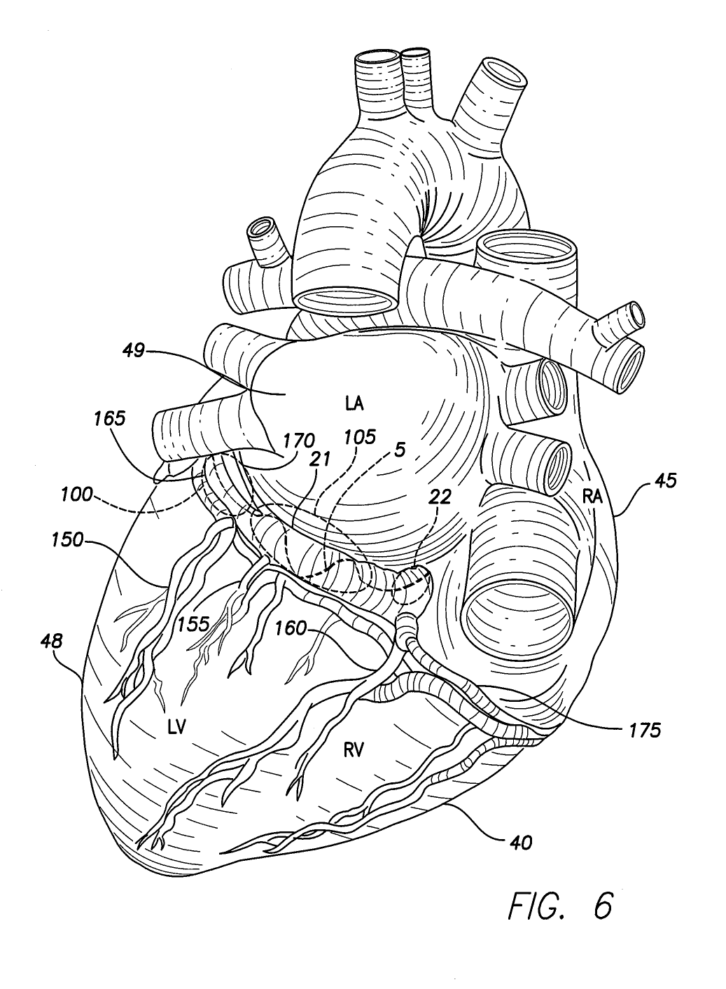

As can be understood from FIG. 6, which is the same view as FIG. 4, in one embodiment, the LV lead 5 can be delivered into the CS 21 and proximal region of the GCV 165 via one or more delivery tools (e.g., stylets, guidewires, sheaths, catheters, etc.). When the delivery tools are removed from the distal portion 50 (see FIGS. 2-3), the humped peaks 115 of the intermediate section 105 of the distal portion 50 of the LV lead 5 are free to bias against the inner wall surface of the CS 21. As a result, each hump peak 115 exerts a force F of between approximately 13,000 dynes to approximately 36,000 dynes for each centimeter the hump peaks 115 are compressed by the inner wall surface of the CS 21 from the free or non-restricted state peak-to-peak distance D1 of between approximately 1.3 cm and approximately 1.5 cm indicated in FIG. 2. The distal section 100 extends into the proximal region of the GCV 165 in a generally linear arrangement with the electrodes 125 oriented so as to generally face the cardiac tissue. For example, the distal section 100 may only extend into the proximal region of the GCV 165 no more than between approximately 2 cm and approximately 7 cm.

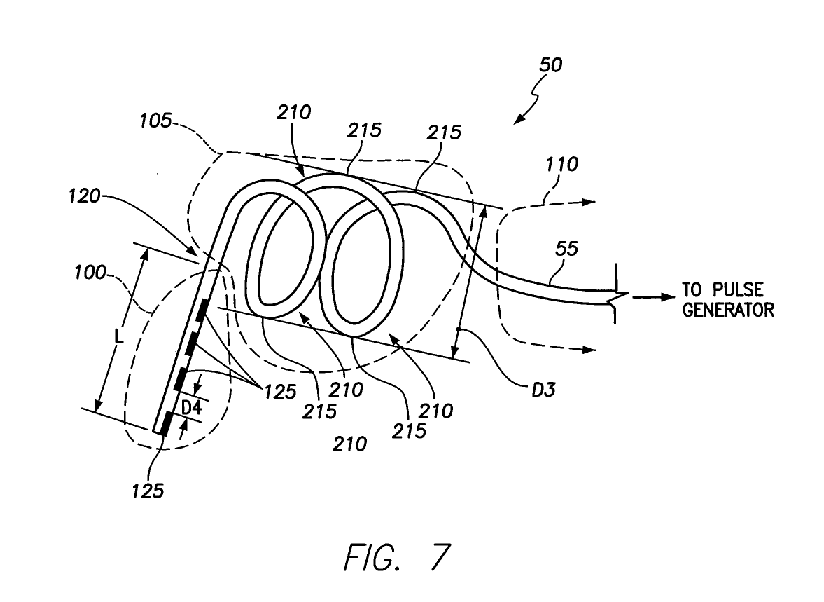

For a discussion of the configuration of the distal portion 50 of a tubular body 55 of a second embodiment of the LV lead 5, reference is made to FIG. 7, which is a side view of the distal portion 50 of the lead tubular body 55 in a free or non-restricted state. As indicated in FIG. 7, when the distal portion 50 of the tubular body 55 of the first embodiment of the LV lead 5 is in a free or non-restricted state, the distal portion 50 includes an extreme distal section 100, an intermediate section 105 immediately proximal the extreme distal section 100, and a proximal section 110 that proximally extends from the intermediate section 105 towards the proximal end of the LV lead 5 that connects to the pulse generator 20, as can be understood from FIG. 1. The tubular body 55 of the intermediate section 105 helically spirals such that the intermediate section 105 has multiple helical coil loops 210 (e.g., two, three or more helical coil loops 210) when in a free or non-restricted state. Each coil loop 210 can be said to have an extreme radially outward point 215 of the coil loop. In a free or non-restricted state, the point-to-point distance D3 between opposite extreme radially outward points 215 is between approximately 1.3 cm and approximately 1.5 cm. The tubular body 55 of the intermediate section 105 has a size of between approximately seven French and approximately eight French and is formed of silicone rubber, polyurethane, or silicone rubber polyurethane copolymer (“SPC”).

As illustrated in FIG. 7, the tubular body 55 of the distal section 100 has a generally straight or linear shape when in a free or non-restricted state. The distal section 100 begins just distal of the most distal coil loop 210 of the intermediate section 105 and extends over a length L of between approximately 1 cm and approximately 3 cm. The tubular body 55 of the distal section 100 has a size of between approximately four French and approximately seven French and is formed of silicone rubber, polyurethane, or silicone rubber polyurethane copolymer (“SPC”). Thus, the transition 120 between the distal section 100 and intermediate section 105 may be in the form of a taper. In other words, the distal section 100 has a tubular body diameter that is smaller than a tubular body diameter of the intermediate section 105, and the lead 5 includes a tubular body diameter transition 120 between the distal section 100 and the intermediate section 105.

As can be understood from FIG. 7, when the distal section 100 and intermediate section 105 are both in a free or non-restricted state, the distal section 100 extends generally perpendicular to a longitudinal axis of the helically coiled configuration of the intermediate section 105.

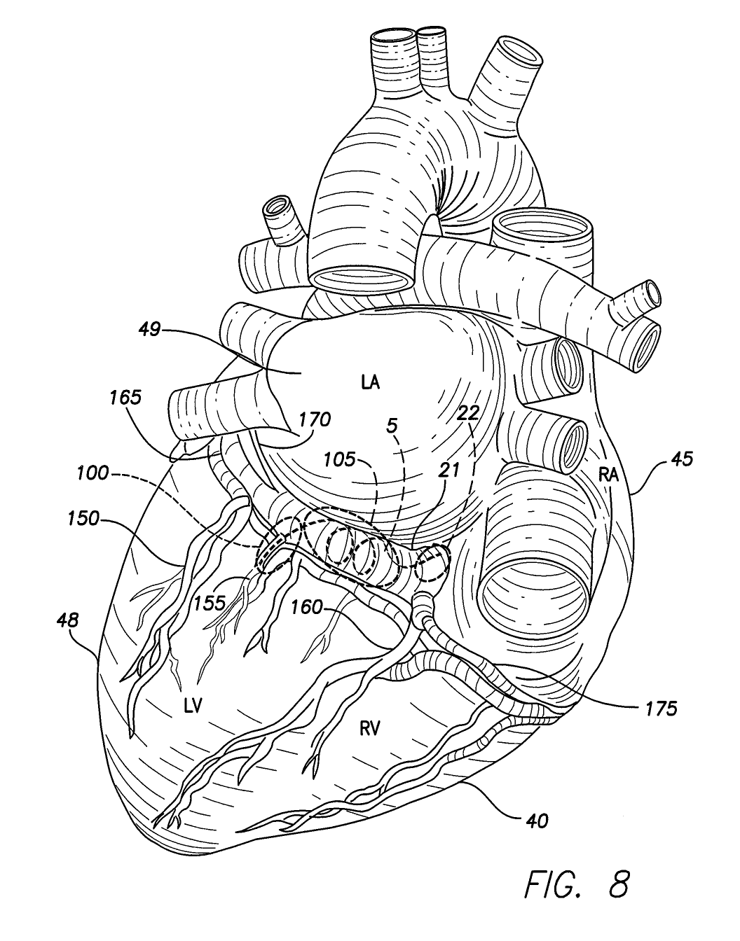

As shown in FIG. 7, the tubular body 55 of the distal section 100 supports multiple electrodes 125 (e.g., two, three, four or more electrodes 125). In one embodiment, there are four electrodes 125 forming a quadpole arrangement wherein the electrodes 125 are spaced apart from each other by a distance D4 of approximately 5 mm. In one embodiment, the electrodes may be positioned on the tubular body 55 so as to be canted (e.g., directed) towards cardiac tissue when the distal portion 50 of the LV lead 5 is deployed as discussed with respect to FIG. 8 below. For example, as can be understood from FIG. 7, in one embodiment, the multiple electrodes 125 of the distal section 100 are located on a side of the tubular body 55 generally oriented proximal. In some embodiments, the electrodes 125 are located on the distal section 100 of the tubular body such that, when looking along the length of the lead from proximal to distal, the multiple electrodes 125 are located on the side of the tubular body 55 such that the multiple electrodes 125 are oriented between facing proximal and facing right. The electrodes 125 being so oriented on the lead body 55 results in the electrodes 125 being directed toward the cardiac tissue, which helps to avoid phrenic nerve stimulation. Also, the electrodes 125 being directed toward the cardiac tissue more efficiently directs the current to the cardiac tissue, thereby minimizing the magnitude of stimulation current needed to pace the cardiac tissue.

In a manner similar to that discussed above with respect to the embodiment of FIG. 3, the body 55 of the distal portion 50 of the LV lead 5 of FIG. 7 is configured to have sufficient bias towards its free or non-restricted state depicted in FIG. 7 such that when confined in and restricted by a CS 21, each extreme radially outward point 215 of a helical coil loop 210 exerts a force of between approximately 13,000 dynes to approximately 36,000 dynes for each centimeter the helical coil loops 210 are compressed from the free or non-restricted state point-to-point distance D3 of between approximately 1.3 cm and approximately 1.5 cm indicated in FIG. 7.

In other words, as can be understood from FIG. 7, when the intermediate section 105 is in the free or non-restricted state, the point-to-point distance D3 extends generally perpendicular to a longitudinal axis of the helically coiled configuration of the intermediate section 105. Further, the point-to-point distance D3 extends between an extreme outward circumferential point 215 of a first loop 210 and an extreme outward circumferential point 215 of a second loop 210 immediately adjacent the first loop 210, the extreme outward circumferential point 215 of the first loop 210 and extreme outward circumferential point 215 of the second loop 210 being on opposite sides of the helically coiled configuration of the intermediate section 105. Thus, when the intermediate section 105 is in the free or non-restricted state, the intermediate section 105 includes a point-to-point distance D3 of between approximately 1.3 cm and approximately 1.5 cm.

As can be understood from FIG. 8, in one embodiment, the LV lead 5 can be delivered into the CS 21 and PCV 155 via one or more delivery tools (e.g., stylets, guidewires, sheaths, catheters, etc.). When the delivery tools are removed from the distal portion 50 (see FIG. 7), the extreme radially outward points 215 of a helical coil loops 210 of the intermediate section 105 of the distal portion 50 of the LV lead 5 are free to bias against the inner wall surface of the CS 21. As a result, each extreme radially outward point 215 exerts a force F of between approximately 13,000 dynes to approximately 36,000 dynes for each centimeter the extreme radially outward points 215 are compressed by the inner wall surface of the CS 21 from the free or non-restricted state point-to-point distance D3 of between approximately 1.3 cm and approximately 1.5 cm indicated in FIG. 7. The distal section 100 extends into the PCV 155 in a generally linear arrangement with the electrodes 125 oriented so as to generally face the cardiac tissue.

In one embodiment, the intermediate section 105 can be secured in the CS 21 as described with respect to FIG. 8. However, instead of extending into the PCV 155, the distal section 100 extends into the LMV 150 in a generally linear arrangement with the electrodes 125 oriented so as to generally face the cardiac tissue.

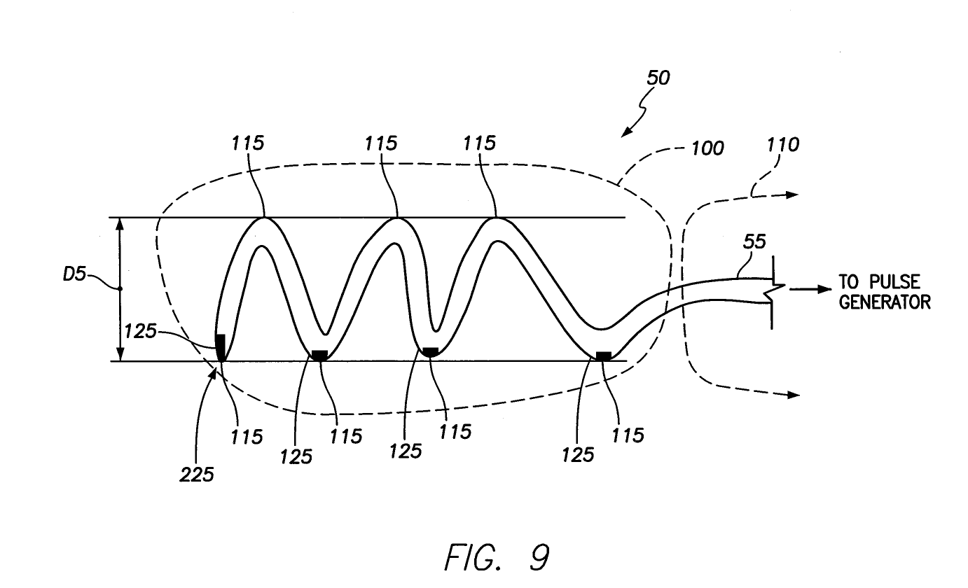

For a discussion of the configuration of the distal portion 50 of a tubular body 55 of a third embodiment of the LV lead 5, reference is made to FIG. 9, which is a side view of the distal portion 50 of the lead tubular body 55 in a free or non-restricted state. As indicated in FIG. 9, when the distal portion 50 of the tubular body 55 of the first embodiment of the LV lead 5 is in a free or non-restricted state, the distal portion 50 includes an extreme distal section 100 and a proximal section 110 that proximally extends from the distal section 100 towards the proximal end of the LV lead 5 that connects to the pulse generator 20, as can be understood from FIG. 1. The tubular body 55 of the distal section 100 undulates in a sinusoidal or S-shaped configuration such that the distal section 100 has multiple hump peaks 115 (e.g., two, three or more hump peaks 115) when in a free or non-restricted state. In a free or non-restricted state, the peak-to-peak distance D5 is between approximately 1.3 cm and approximately 1.5 cm.

In other words, as can be understood from FIG. 9, when the distal section 100 is in the free or non-restricted state and the proximal section 110 is laid out in a straight line, the peak-to-peak distance D5 extends generally perpendicular to a longitudinal axis of the straight proximal section 110. Further, the peak-to-peak distance D5 extends between the peak 115 of a first hump or bend and a peak 115 of a second hump or bend immediately adjacent the first hump or bend and projecting in a direction opposite the first hump or bend. Thus, when the distal section 100 is in the free or non-restricted state as depicted in FIG. 9, the distal section 100 includes a peak-to-peak distance D5 of between approximately 1.3 cm and approximately 1.5 cm.

The tubular body 55 of the distal section 100 has a size of between approximately seven French and approximately eight French and is formed of silicone rubber, polyurethane, or silicone rubber polyurethane copolymer (“SPC”). The distal section 100 terminates distally at a free distal end 225, which may extend from the most distal hump peak 115 to a point approximately even with the second most distal hump peak 115.

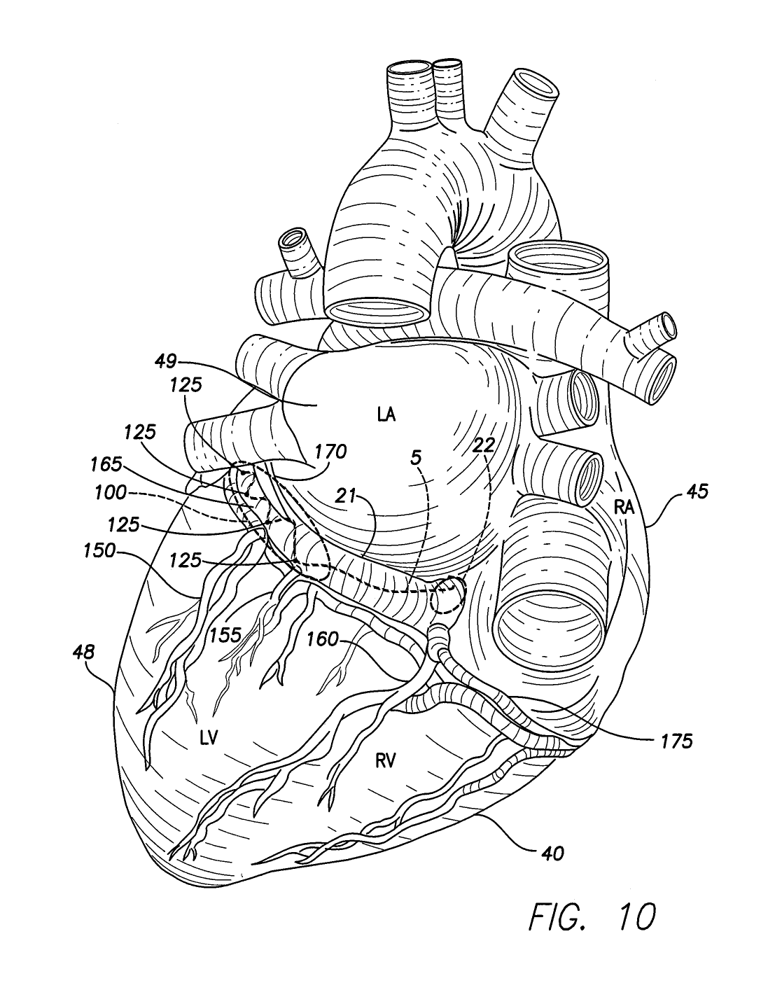

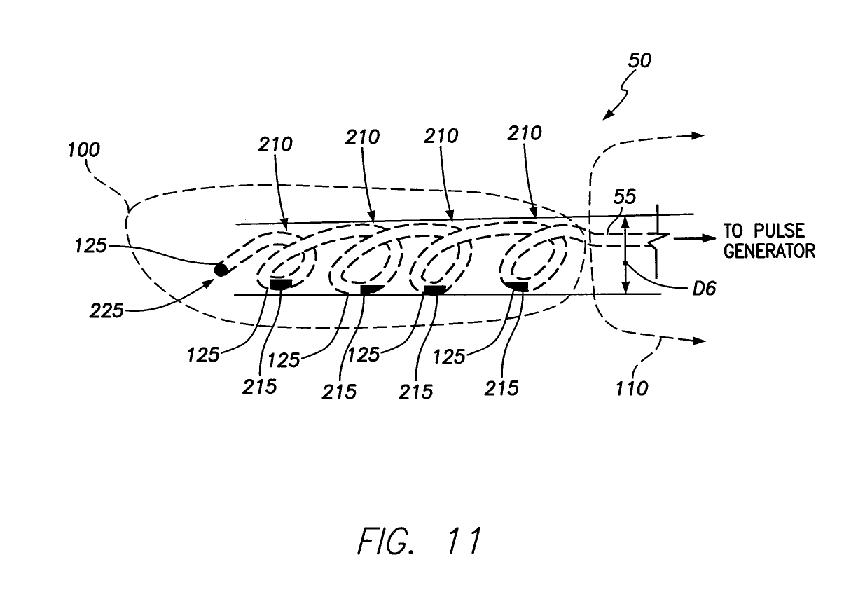

As shown in FIG. 9, the tubular body 55 of the distal section 100 supports multiple electrodes 125 (e.g., two, three, four or more electrodes 125). In one embodiment, there are four electrodes 125 forming a quadpole arrangement. As indicated in FIG. 9, in one embodiment, an individual electrode 125 may be located at each respective hump peak 115 and the free distal end 225 on the side of the distal section 100 corresponding to the free distal end 225. In one embodiment, the electrodes may be positioned on the tubular body 55 so as to be canted (e.g., directed) towards cardiac tissue when the distal portion 50 of the LV lead 5 is deployed as discussed with respect to FIG. 10 below. For example, as can be understood from FIG. 9, in one embodiment, the multiple electrodes 125 of the distal section 100 are located on the tubular body 55 generally oriented in the direction the hump peaks 115 are projecting. In some embodiments, the electrodes 125 are located on the distal section 100 of the tubular body such that, when looking along the length of the lead 5 from proximal to distal, the electrodes 125 are located on the tubular body 55 such that the electrodes 125 are oriented between facing in the direction the hump peaks 115 are projecting and facing right. The electrodes 125 being so oriented on the lead body 55 results in the electrodes 125 being directed toward the cardiac tissue, which helps to avoid phrenic nerve stimulation. Also, the electrodes 125 being directed toward the cardiac tissue more efficiently directs the current to the cardiac tissue, thereby minimizing the magnitude of stimulation current needed to pace the cardiac tissue.

In a manner similar to that discussed above with respect to the embodiment of FIG. 3, the body 55 of the distal portion 50 of the LV lead 5 of FIG. 9 is configured to have sufficient bias towards its free or non-restricted state depicted in FIG. 9 such that when confined in and restricted by a CS 21, each hump peak 115 exerts a force of between approximately 13,000 dynes to approximately 36,000 dynes for each centimeter the hump peaks 115 are compressed from the free or non-restricted state peak-to-peak distance D5 of between approximately 1.3 cm and approximately 1.5 cm indicated in FIG. 9.

As can be understood from FIG. 10, in one embodiment, the LV lead 5 can be delivered into the CS 21 and GCV 165 via one or more delivery tools (e.g., stylets, guidewires, sheaths, catheters, etc.). When the delivery tools are removed from the distal portion 50 (see FIG. 9), the hump peaks 115 of the distal section 100 of the distal portion 50 of the LV lead 5 are free to bias against the inner wall surface of the CS 21. As a result, each hump peak 115 exerts a force of between approximately 13,000 dynes to approximately 36,000 dynes for each centimeter the hump peaks 115 are compressed by the inner wall surface of the CS 21 from the free or non-restricted state peak-to-peak distance D5 of between approximately 1.3 cm and approximately 1.5 cm indicated in FIG. 9. The distal section 100 extends into the GCV 165 in the sinusoidal or humped arrangement with the electrodes 125 oriented so as to generally face the cardiac tissue. In such an arrangement where the electrode equipped portion of the distal section 100 extends through the CS 21 and GCV 165, the electrodes can stimulate a large portion of the inferior region of the distal coronary sinus 21. Programming or auto-threshold techniques can be used to determine which electrodes should be activated to stimulate the base of the LV 48 with a low threshold.

For a discussion of the configuration of the distal portion 50 of a tubular body 55 of a third embodiment of the LV lead 5, reference is made to FIG. 11, which is a side view of the distal portion 50 of the lead tubular body 55 in a free or non-restricted state. As indicated in FIG. 11, when the distal portion 50 of the tubular body 55 of the first embodiment of the LV lead 5 is in a free or non-restricted state, the distal portion 50 includes an extreme distal section 100 and a proximal section 110 that proximally extends from the distal section 100 towards the proximal end of the LV lead 5 that connects to the pulse generator 20, as can be understood from FIG. 1. The tubular body 55 of the distal section 100 helically spirals such that the distal section 100 has multiple helical coil loops 210 (e.g., two, three or more helical coil loops 210) when in a free or non-restricted state. Each coil loop 210 can be said to have an extreme radially outward point 215 of the coil loop 210. In a free or non-restricted state, the point-to-point distance D6 between opposite extreme radially outward points 215 is between approximately 1.3 cm and approximately 1.5 cm. The tubular body 55 of the distal section 100 has a size of between approximately seven French and approximately eight French and is formed of silicone rubber, polyurethane, or silicone rubber polyurethane copolymer (“SPC”). The distal section 100 terminates distally at a free distal end 225, which may extend from the most distal helical coil loop 210 to a point approximately even with the most distal extreme radially outward point 215 of the most distal coil loop 210.

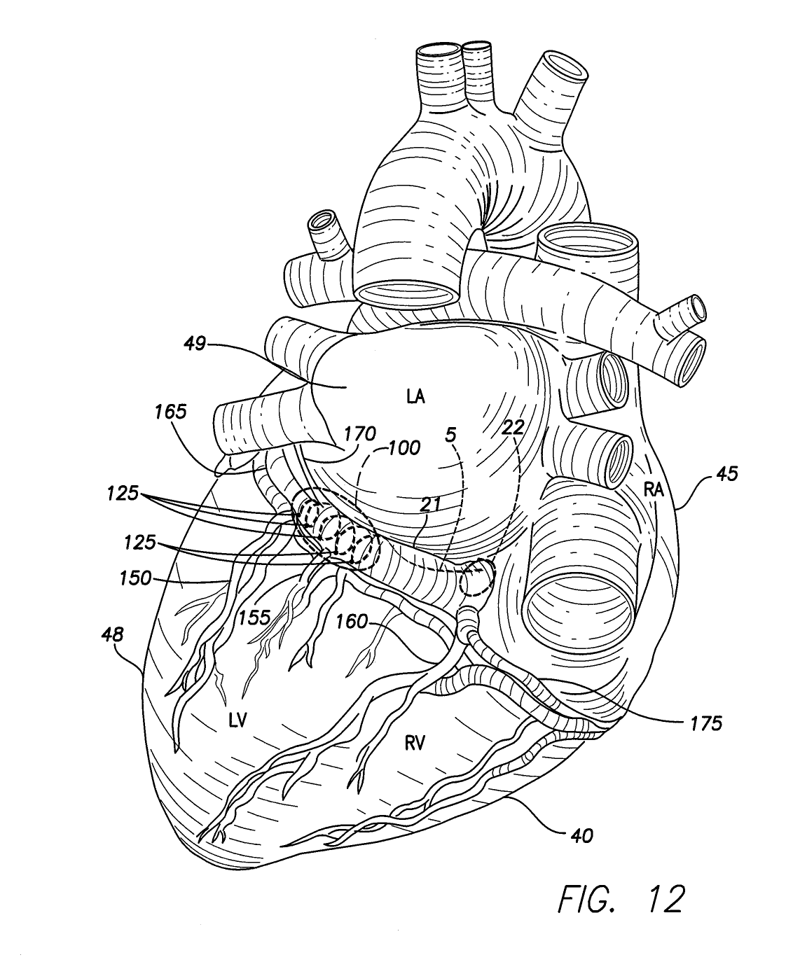

As shown in FIG. 11, the tubular body 55 of the distal section 100 supports multiple electrodes 125 (e.g., two, three, four or more electrodes 125). In one embodiment, there are five electrodes 125. As indicated in FIG. 11, in one embodiment, an individual electrode 125 may be located at each respective extreme radially outward point 215 of a coil loop 210 and the free distal end 225 on the side of the distal section 100 corresponding to the free distal end 225. In one embodiment, the electrodes 125 may be positioned on the tubular body 55 so as to be canted (e.g., directed) towards cardiac tissue when the distal portion 50 of the LV lead 5 is deployed as discussed with respect to FIG. 12 below. For example, as can be understood from FIG. 11, in one embodiment, the electrodes 125 of the distal section 100 are located on the tubular body 55 generally oriented in the direction the distal free end 225 is projecting. In some embodiments, the electrodes 125 are located on the distal section 100 of the tubular body such that, when looking along the length of the lead 5 from proximal to distal, the electrodes 125 are located on the tubular body 55 such that the electrodes 125 are oriented between facing in the direction the distal free end 225 is projecting and facing right. The electrodes 125 being so oriented on the lead body 55 results in the electrodes 125 being directed toward the cardiac tissue, which helps to avoid phrenic nerve stimulation. Also, the electrodes 125 being directed toward the cardiac tissue more efficiently directs the current to the cardiac tissue, thereby minimizing the magnitude of stimulation current needed to pace the cardiac tissue.

In a manner similar to that discussed above with respect to the embodiment of FIG. 3, the body 55 of the distal portion 50 of the LV lead 5 of FIG. 11 is configured to have sufficient bias towards its free or non-restricted state depicted in FIG. 11 such that when confined in and restricted by a CS 21, each extreme radially outward point 215 of a helical coil loop 210 exerts a force of between approximately 13,000 dynes to approximately 36,000 dynes for each centimeter the helical coil loops 210 are compressed from the free or non-restricted state point-to-point distance D6 of between approximately 1.3 cm and approximately 1.5 cm indicated in FIG. 11.

In other words, as can be understood from FIG. 11, when the distal section 100 is in the free or non-restricted state, the point-to-point distance D6 extends generally perpendicular to a longitudinal axis of the helically coiled configuration of the distal section 100. Further, the point-to-point distance D6 extends between an extreme outward circumferential point 215 of a first loop 210 and an extreme outward circumferential point 215 of a second loop 210 immediately adjacent the first loop 210, the extreme outward circumferential point 215 of the first loop 210 and extreme outward circumferential point 215 of the second loop 210 being on opposite sides of the helically coiled configuration of the distal section 100. Thus, when the distal section 100 is in the free or non-restricted state, the distal section 100 includes a point-to-point distance D6 of between approximately 1.3 cm and approximately 1.5 cm.

As can be understood from FIG. 12, in one embodiment, the LV lead 5 can be delivered into the CS 21 via one or more delivery tools (e.g., stylets, guidewires, sheaths, catheters, etc.). When the delivery tools are removed from the distal portion 50 (see FIG. 11), the extreme radially outward points 215 of a helical coil loops 210 of the intermediate section 105 of the distal portion 50 of the LV lead 5 are free to bias against the inner wall surface of the CS 21. As a result, each extreme radially outward point 215 exerts a force of between approximately 13,000 dynes to approximately 36,000 dynes for each centimeter the extreme radially outward points 215 are compressed by the inner wall surface of the CS 21 from the free or non-restricted state point-to-point distance D6 of between approximately 1.3 cm and approximately 1.5 cm indicated in FIG. 11. The implantation of FIG. 12 can be used to stimulate the base of the LV 48.

While in one embodiment shown in FIG. 12 the distal section 100 of the LV lead 5 is confined generally to the CS 21, in other embodiments, the distal section 100 of the LV lead 5 may be located in both the CS 21 and proximal region of the GCV 165 similar to that of FIG. 10.

As can be understood from FIGS. 2-8, in some embodiments, the distal portion 50 of the LV lead 5 includes stabilization features (e.g., S-shaped or helical shaped) for stabilizing the lead distal portion 50 in the coronary sinus 21, the lead distal portion 50 distally terminating in a linearly straight segment 100 configured for placement in a coronary vein tributary near the base of the LV 48.

As can be understood from FIGS. 9-12, in some embodiments, the distal portion 50 of the LV lead 5 includes stabilization features (e.g., S-shaped or helical shaped) for stabilizing the lead distal portion 50 in the coronary sinus 21, the lead distal portion 50 supporting electrodes oriented to stimulate the inferior portion of the CS 21 to pace the base of the LV 48.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. An implantable medical lead for coupling to an implantable pulse generator and targeted stimulation of the lateral and posterior basal left ventricular region of a patient heart, the lead comprising a tubular body comprising:
an intermediate section that biases into a generally S-shaped or sinusoidal-shaped configuration when the intermediate section is in a free or non-restricted state, the S-shaped or sinusoidal-shaped configuration comprising multiple hump peaks comprising at least a most distal hump peak and a most proximal hump peak;
a proximal section proximally extending from the most proximal hump peak to a proximal end configured to electrically couple to the implantable pulse generator; and
a distal section that biases into a generally straight linear shaped configuration when the distal section is in a free or non-restricted state, the distal section distally extending from the most distal hump peak and comprising multiple electrodes and a distal free end that forms an extreme distal end of the lead.

2. The lead of claim 1, wherein the multiple electrodes are spaced apart from each other by a distance of approximately 5 mm.

3. The lead of claim 1, wherein the distal section has a tubular body diameter that is smaller than a tubular body diameter of the intermediate section, and the lead further comprises a tubular body diameter transition between the distal section and the intermediate section.

4. The lead of claim 3, wherein the distal section has a length of between approximately 1 cm and approximately 3 cm between the distal free end and the tubular body diameter transition.

5. The lead of claim 3, wherein the tubular body diameter of the distal section is between approximately four French and approximately seven French, and the tubular body diameter of the intermediate section is between approximately seven French and eight French.

6. The lead of claim 4, wherein, when the intermediate section is in the free or non-restricted state, the intermediate section includes a peak-to-peak distance of between approximately 1.3 cm and approximately 1.5 cm, wherein the peak-to-peak distance extends generally perpendicular to a longitudinal axis of the proximal section when the proximal section is laid out in a straight line, the peak-to-peak distance extending between a first hump peak and a second hump peak immediately adjacent the first hump peak and projecting in a direction opposite the first hump peak.

7. The lead of claim 6, wherein the first hump peak and second hump peak each exert a force of between approximately 13,000 dynes to approximately 36,000 dynes for each centimeter the hump peak is compressed from the free or non-restricted state peak-to-peak distance.

8. The lead of claim 1, wherein, when the distal section and intermediate section are both in a free or non-restricted state, the tubular body forming the intermediate section and distal section generally resides in a single plane.

9. The lead of claim 8, wherein the multiple electrodes of the distal section are located on a side of the tubular body generally oriented proximal.

10. The lead of claim 9, wherein, when looking along the length of the lead from proximal to distal, the multiple electrodes are located on the side of the tubular body such that the multiple electrodes are oriented between facing proximal and facing right.

11. An implantable medical lead for coupling to an implantable pulse generator and targeted stimulation of the lateral and posterior basal left ventricular region of a patient heart, the lead comprising a tubular body comprising:
an intermediate section that biases into a generally helically coiled configuration when the intermediate section is in a free or non-restricted state, the helically coiled configuration comprising multiple helical coil loops comprising at least a most distal loop and a most proximal loop;
a proximal section proximally extending from the most proximal loop to a proximal end configured to electrically couple to the implantable pulse generator; and
a distal section that biases into a generally straight linear shaped configuration when the distal section is in a free or non-restricted state, the distal section distally extending from the most distal loop and comprising multiple electrodes and a distal free end that forms an extreme distal end of the lead.

12. The lead of claim 11, wherein the multiple electrodes are spaced apart from each other by a distance of approximately 5 mm.

13. The lead of claim 11, wherein the distal section has a tubular body diameter that is smaller than a tubular body diameter of the intermediate section, and the lead further comprises a tubular body diameter transition between the distal section and the intermediate section.

14. The lead of claim 13, wherein the distal section has a length of between approximately 1 cm and approximately 3 cm between the distal free end and the tubular body diameter transition.

15. The lead of claim 13, wherein the tubular body diameter of the distal section is between approximately four French and approximately seven French, and the tubular body diameter of the intermediate section is between approximately seven French and eight French.

16. The lead of claim 14, wherein, when the intermediate section is in the free or non-restricted state, the intermediate section includes a point-to-point distance of between approximately 1.3 cm and approximately 1.5 cm, wherein the point-to-point distance extends generally perpendicular to a longitudinal axis of the helically coiled configuration, the point-to-point distance extending between an extreme outward circumferential point of a first loop and an extreme outward circumferential point of a second loop immediately adjacent the first loop, the extreme outward circumferential point of the first loop and extreme outward circumferential point of the second loop being on opposite sides of the helically coiled configuration.

17. The lead of claim 16, wherein the extreme outward circumferential point of the first loop and extreme outward circumferential point of the second loop each exert a force of between approximately 13,000 dynes to approximately 36,000 dynes for each centimeter the helically coiled configuration is compressed from the free or non-restricted state point-to-point distance.

18. The lead of claim 11, wherein, when the distal section and intermediate section are both in a free or non-restricted state, the distal section extends generally perpendicular to a longitudinal axis of the helically coiled configuration.

19. The lead of claim 18, wherein the multiple electrodes of the distal section are located on a side of the tubular body generally oriented proximal.

20. The lead of claim 19, wherein, when looking along the length of the lead from proximal to distal, the multiple electrodes are located on the side of the tubular body such that the multiple electrodes are oriented between facing proximal and facing right.

21. An implantable medical lead for coupling to an implantable pulse generator and targeted stimulation of the lateral and posterior basal left ventricular region of a patient heart, the lead comprising a tubular body comprising:
a distal section that biases into a generally S-shaped or sinusoidal-shaped configuration when the distal section is in a free or non-restricted state, the S-shaped or sinusoidal-shaped configuration comprising multiple hump peaks comprising at least a most distal hump peak and a most proximal hump peak, the most distal hump peak extending into a distal termination of the S-shaped or sinusoidal-shaped configuration in the form of a distal free end that forms an extreme distal end of the lead, the distal section comprising first, second, third and fourth electrodes, the first electrode supported on the tubular body at the distal free end, the second, third and fourth electrodes supported on the tubular body at respective hump peaks located on the same side of the distal section as the distal free end; and
a proximal section proximally extending from the most proximal hump peak to a proximal end configured to electrically couple to the implantable pulse generator.

22. The lead of claim 21, wherein the tubular body diameter of the distal section is between approximately seven French and eight French.

23. The lead of claim 22, wherein, when the distal section is in the free or non-restricted state, the distal section includes a peak-to-peak distance of between approximately 1.3 cm and approximately 1.5 cm, wherein the peak-to-peak distance extends generally perpendicular to a longitudinal axis of the proximal section when the proximal section is laid out in a straight line, the peak-to-peak distance extending between a first hump peak and a second hump peak immediately adjacent the first hump peak and projecting in a direction opposite the first hump peak.

24. The lead of claim 23, wherein the first hump peak and second hump peak each exert a force of between approximately 13,000 dynes to approximately 36,000 dynes for each centimeter the hump peak is compressed from the free or non-restricted state peak-to-peak distance.

25. The lead of claim 21, wherein the electrodes of the distal section are located on the tubular body generally oriented in the direction the hump peaks are projecting.

26. The lead of claim 21, wherein, when looking along the length of the lead from proximal to distal, the electrodes are located on the tubular body such that the electrodes are oriented between facing in the direction the hump peaks are projecting and facing right.

27. An implantable medical lead for coupling to an implantable pulse generator and targeted stimulation of the lateral and posterior basal left ventricular region of a patient heart, the lead comprising a tubular body comprising:
a distal section that biases into a generally helically coiled configuration when the distal section is in a free or non-restricted state, the helically coiled configuration comprising multiple helical coil loops comprising at least a most distal loop and a most proximal loop, the most distal loop extending into a distal termination of the helically coiled configuration in the form of a distal free end that forms an extreme distal end of the lead, the distal section comprising first, second, third and fourth electrodes, the first electrode supported on the tubular body at the distal free end, the second, third and fourth electrodes each supported on the tubular body at an extreme outward circumferential point of a respective loop, each of the second, third and fourth electrodes located on the same side of the distal section as the distal free end; and
a proximal section proximally extending from the most proximal loop to a proximal end configured to electrically couple to the implantable pulse generator.

28. The lead of claim 27, wherein the tubular body diameter of the distal section is between approximately seven French and eight French.

29. The lead of claim 28, wherein, when the distal section is in the free or non-restricted state, the distal section includes a point-to-point distance of between approximately 1.3 cm and approximately 1.5 cm, wherein the point-to-point distance extends generally perpendicular to a longitudinal axis of the helically coiled configuration, the point-to-point distance extending between an extreme outward circumferential point of a first loop and an extreme outward circumferential point of a second loop immediately adjacent the first loop, the extreme outward circumferential point of the first loop and extreme outward circumferential point of the second loop being on opposite sides of the helically coiled configuration.

30. The lead of claim 29, wherein the extreme outward circumferential point of the first loop and extreme outward circumferential point of the second loop each exert a force of between approximately 13,000 dynes to approximately 36,000 dynes for each centimeter the helically coiled configuration is compressed from the free or non-restricted state point-to-point distance.

31. The lead of claim 27, wherein the electrodes of the distal section are located on the tubular body generally oriented in the direction the distal free end is projecting.

32. The lead of claim 27, wherein, when looking along the length of the lead from proximal to distal, the electrodes are located on the tubular body such that the electrodes are oriented between facing in the direction the distal free end is projecting and facing right.
